(19) United States
(12) Patent Application Publication
(10) Pub. No.: US 2010/0161003 A1
(43) Pub. Date: Jun. 24, 2010

(54) IMPLANTABLE MEDICAL DEVICE

(76) Inventors: Patrik Malmberg, Stockholm (SE); Jürgen Kerstna, Hasselby (SE)

Correspondence Address: SCHIFF HARDIN, LLP;PATENT DEPARTMENT, 233 S. Wacker Drive-Suite 6600, CHICAGO, IL, 60606-6473, US

(21) Appl. No.: 12/600,558

(22) PCT Filed: May 28, 2007
(86) PCT No.: PCT/SE07/00512

§ 371 Date: Nov. 17, 2009

(51) Int. Cl. A61N 1/08; G08B 23/00

(52) U.S. Cl. 607/60; 340/573.1

(57) ABSTRACT

An implantable medical device has a programmer for generating a programming command representative of a diagnostic status of a subject or an operation status of the medical device. The device also comprises an interface for communicating the command to a programmable, implantable RFID tag. The tag memory is reprogrammed based on the received command. A portable RFID reader is used for interrogating the RFID tag and data in the tag memory can be displayed on the reader.

### BACKGROUND OF THE INVENTION

1. Field of the Invention

The present invention generally relates to an implantable medical device, and in particular to such a medical device connected to a radio frequency identification tag.

2. Description of the Prior Art

People with severe diseases that can occur at any time often carry a card describing their medical conditions. For example, patients having a patient history of heart attacks are typically prescribed agents reducing the coagulability of the blood. These patients often carry cards with information of the prescribed agent. Patients suffering from diabetes, likewise often carry a card or pendant specifying that they are diabetic, which information is of high value for medical personnel in the case of an emergency situation involving the patient. A further example is patients with pacemakers, defibrillators and other implantable medical devices.

In these prior art cases, the card or pendant merely expresses the type of medicament that the patient takes or that the patient has an implantable medical device, for instance. However, in the case of an emergency occasion involving the patient, the card does not provide any further descriptive information of the actual condition resulting in the life-threatening situation. This means that the information provided on the card will be of limited use to a person finding the patient severely affected or even unconscious.

It has been suggested in the art that in response to an emergency situation, an implantable medical device compiles and transmits a message describing the life-threatening situation. This message is typically first transmitted to a portable programmer or personal digital assistant (PDA) carried by the patient. This PDA then forwards the message to an emergency central of a healthcare enterprise, alerting the medical personnel of the situation. However, in such a case, this descriptive information is not available to persons in the surrounding of the patient. Furthermore, there may be occasional mistakes in the forwarding of the received information from the emergency central to the actual medical personnel that will first reach the patient. The personnel have then only access to the limited information provided by the card worn by the patient. This way of informing medical personnel is further dependent on the continuous carrying of the portable PDA by the patient. Thus, if the patient forgets to bring the PDA along, no notification to medical personnel is possible at all in the case of an emergency situation.

### SUMMARY OF THE INVENTION

The present invention overcomes these and other drawbacks of the prior art arrangements.

It is a general object of the present invention to provide an implantable medical device having RFID programming functionality.

It is another object of the invention to provide a system based on an implantable medical device and an implantable RFID tag.

It is a particular object of the invention to provide a condition system that can be used for displaying informative data in the case of a severe medical condition or device malfunctioning.

Briefly, the present invention involves a system having an implantable medical device (IMD) and an implantable radio frequency identification (RFID) tag with a readable and writable tag memory. The IMD collects and processes diagnostic data measured in a subject body for the purpose of generating different diagnostic parameters. These parameters are descriptive of the medical status of the subject and can be used for detecting different, more or less severe, medical conditions. In addition, the IMD preferably also collects data of its operation, such as battery status, lead impedance of cardiac leads employed for cardiac pacing or defibrillation, etc. Such data is also processed by the IMD for the purpose of generating different operation status parameters. An RFID programmer implemented in the IMD uses the generated diagnostic and/or status parameters for generating a programming command. This command is forwarded to the implantable RFID tag, where the tag memory is reprogrammed based on the received command.

The subject also has a portable smart card or unit with an integrated RFID reader that can generate a tag memory interrogation urging the RFID tag to transmit the data stored in its tag memory. This data can then be presented on a display screen of the smart card or be forwarded to another connected screen.

The presented data can include information of subject, such a subject name, and information of the IMD, such as IMD type, model and serial number. In addition, information descriptive of diagnostic and status parameters is displayed on the screen. This information is very important in the case of an emergency situation, for example when the patient is having a severe medical condition or the IMD is starting to malfunction. The subject can then call an emergency center or his/her physician and inform them of the data presented on the display screen. Also time stamping of the data representing the time and date in which it was generated or entered in the rag memory can be of relevance.

In a preferred embodiment, the IMD also generates action information based on the diagnostic and/or status parameter(s). This information is descriptive of a preferred action to be taken by the subject or some other person viewing the screen. In such a case, the programming command also comprises such action information. When displayed on the screen, the information can urge a reading person to perform special actions such as giving first aid, administering some medicaments and/or calling an emergency center.

The present invention provides a system and method for communicating more informative data to an IMD subject or a person finding the subject unconscious or severely affect. This informative data is highly useful by medical personnel that are to treat the subject in the case of an emergency situation and allows a correct treatment to be initiated directly without the need of extensive diagnosis for determining the cause of the subject's medical condition.

Other advantages offered by the present invention will be appreciated upon reading of the below description of the embodiments of the invention.

### BRIEF DESCRIPTION OF THE DRAWINGS

FIG. 1 is a schematic overview of a condition system of the present invention in operation, partly implanted in a subject.

FIG. 2 is a schematic block diagram of an embodiment of an implantable medical device according to the present invention.

FIG. 3 is a schematic block diagram of the event detector of FIG. 2 in more detail according to an embodiment of the present invention.

FIG. 4 is a schematic block diagram of an embodiment of an implantable RFID tag according to the present invention.

FIG. 5 is a schematic block diagram of another embodiment of an implantable medical device according to the present invention.

FIG. 6 is a schematic block diagram of an embodiment of an RFID reader according to the present invention.

FIG. 7 is a schematic block diagram of an embodiment of a mobile communications unit according to the present invention.

FIG. 8 is a flow diagram of a condition method according to the present invention.

FIG. 9 is a flow diagram illustrating additional steps of the condition method of FIG. 8.

FIG. 10 is a flow diagram illustrating additional steps of the condition method of FIG. 8.

FIG. 11 is a flow diagram illustrating additional steps to the condition method of FIG. 8.

FIG. 12 is a flow diagram illustrating additional steps to the condition method of FIG. 11.

### DESCRIPTION OF THE PREFERRED EMBODIMENTS

The present invention relates to systems and methods that allow display of condition or status data of a subject, preferably human subject, having an implantable medical device. This status data could be of informative character to the subject, for instance by specifying that the operation status of the medical device is correct and/or that no severe medical condition has been detected by the device. Furthermore, information of change in the operation mode or parameters of the implantable medical device could be status data of informative characters. However, the present invention is in particular advantageous if a divergence to the normal operation status is taking place or a potentially harmful medical condition is detected. The invention then allows display of descriptive information to the subject and to medical personnel and other persons finding the subject severely affected or even unconscious.

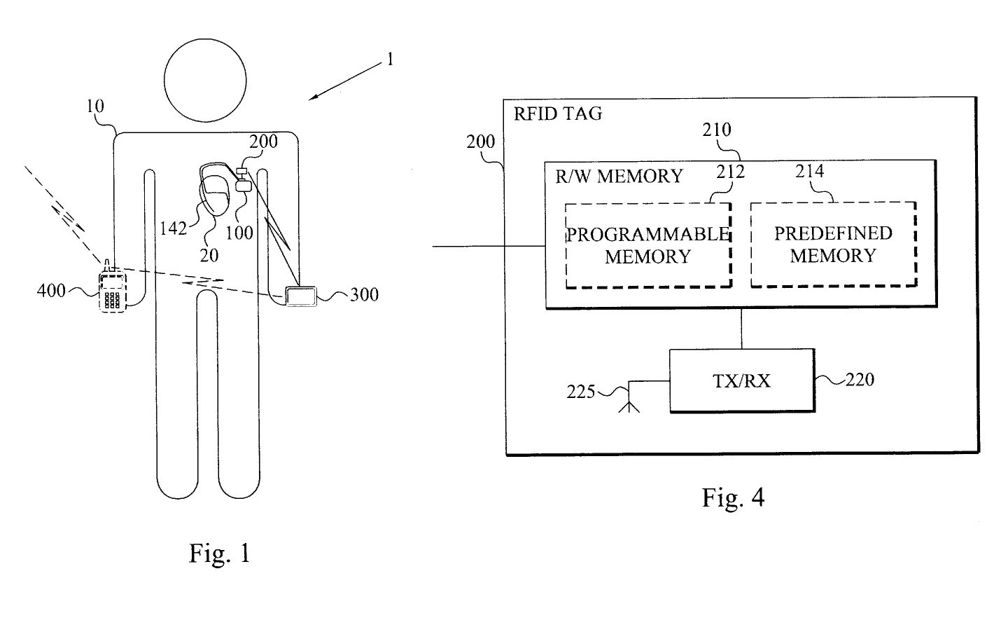

FIG. 1 is a schematic overview of the key units in the condition system 1 of the present invention. The system 1 includes an implantable medical device (IMD) 100 implantable into a subject 10, preferably a human subject. The IMD 100 is illustrated as a device that monitors and/or provides therapy to the heart 20 of the patient 10, such as a pacemaker, defibrillator or cardioverter. The IMD 100 therefore has a connected lead 142 with electrodes and/or sensors for performing the therapy and/or monitor function to the heart 20. However, the present invention is not limited to cardiac-associated IMDs but may also be practiced with other implantable medical devices 100, such as drug pumps, neurological stimulators, physical signal recorders, oxygen sensors, or the like.

According to the invention this IMD 100 comprises a radio frequency identification (RFID) programmer that is able to program a readable and writable memory of an implantable RFID tag or RFID-based near field communication unit 200. This RFID tag 200 can be integrated in the IMD 100 or be wirelessly or hardwired connected to the IMD 100 inside the subject's body 10. The IMD 100 is thereby able to program the memory with condition information, including such information descriptive of the operational status of the IMD 100 and the current medical status of the subject 10.

An external RFID reader or interrogator 300 is carried or worn by the subject 10. The RFID reader 300 will, upon transmission of a memory interrogation request to the RFID tag 200, receive the data currently stored in the tag memory. The received condition information can therefore be displayed on the RFID reader 300 itself and/or may be transmitted to another external display unit 400, represented by a portable user terminal (mobile telephone) 400 in FIG. 1. In either case, the displayed data is of informative purpose to the subject 10 or other persons viewing the display screen, in particular in connection with an emergency event where the subject 10 can be unconscious or otherwise unable to communicate with others.

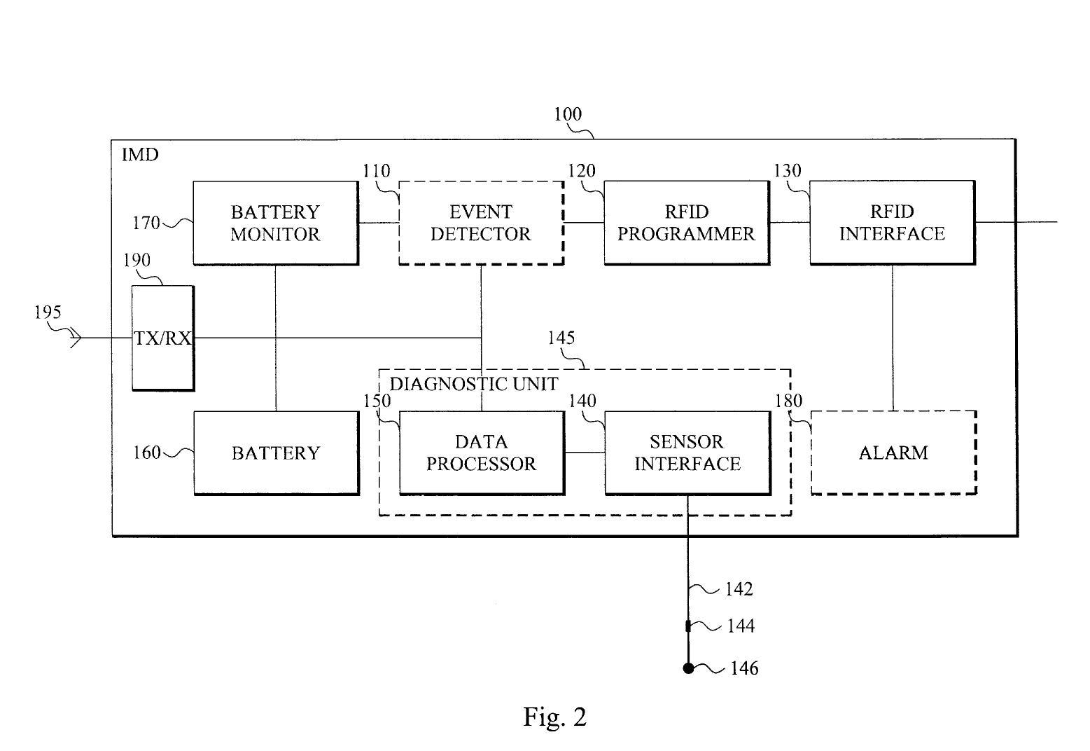

FIG. 2 is a schematic block diagram of an embodiment of an IMD 100 according to the present invention. The IMD 100 comprises an RFID programmer 120 arranged for generating a programming command that can be used for reprogramming a connected implanted RFID tag. This programming command is indicative of condition information associated with the IMD 100 or with the subject, in which the IMD 100 is implanted during operation. In a particular preferred implementation, this condition information is representative of a diagnostic status of the subject and/or an operation status of the IMD 100.

The generated programming command is forwarded to an RFID interface 130 of the IMD 100. This interface 130 is implemented in the IMD 100 for handling any communication between the IMD 100 and the RFID tag. In a first implementation, the RFID interface 130 has a wired connection to the RFID tag as is schematically illustrated in FIG. 2. In such a case, the interface 130 basically forwards the programming command from the programmer 120 to the connected tag for rewriting the tag memory based on the command. However, in a second possible implementation, the interface 130 is in wireless communication with the RFID tag. The interface 130 therefore comprises, or is connected to, dedicated functionality and equipment for enabling this communication, such as antenna, modulator, encoder, etc. This wireless communication can be affected by, for instance, RF-based, IR-based, induction-based near field communications technique well known in the art.

The input to the RFID programmer 120 is generated by other units of the IMD 100. For example, condition and diagnostic status of the subject is determined by a diagnostic unit 145. In a preferred implementation, this diagnostic unit comprises a sensor interface 140 and a data processor 150.

The sensor interface 140 is, in operation, connected to a sensor unit 144, 146 provided in the subject body for the purpose of performing diagnostic measurements of different quantities. For example, the sensor interface 140 can be connected to a lead 142 with electrodes 144, 146 that is used for providing pacing or shocking therapy to the subject when needed. The electrodes 144, 146 of the lead 142 can also be used for bioimpedance measurements in or in connection of the subject's heart. The collected impedance data is collected by the interface 140 and forwarded to the data processor 150 for further processing to be useful as diagnostic status basis.

Cardiac impedance measurements should merely be seen as an illustrative example of possible diagnostic data that can be collected by sensors 144, 146 connected to the sensor interface 140. Actually any implantable sensors known in the art and that could be of diagnostic use for the IMD subject can be employed according to the present invention. Non-limiting examples of such sensors include impedance sensors, oxygen sensors, pressure sensors, temperature sensors, activity sensors, posture sensors, acceleration sensors, stretch sensors, optical sensors, microphones, vibration sensors, sensors dedicated for measuring a particular agent or substance in the subject body, such as glucose sensors and sensors for sodium, potassium, carbon dioxide, chloride, creatinine, blood urea nitrogen (BUN), high-density lipoprotein, low-density lipoprotein, bilirubin, etc.

The measured raw sensor data is brought to the data processor 150 of the diagnostic unit 145. This data processor 150 processes the data in order to generate a diagnostic parameter that is representative of the condition or diagnostic status of the subject. For example, the data processor 150 could include a set of filters, dividers, integrators, etc. that can be applied to the raw data to obtain cardiac impedance, respiratory impedance and/or direct current impedance based on information of an applied current signal and a resulting voltage response measured over the sensors 144, 146.

Operation status of the IMD 100 can also be collected by the diagnostic unit 145. For example, the sensors 144, 146 on the lead 142 connected to the interface 140 can be used for estimating the lead impedance. This impedance is a measure of how well the lead 142 is connected to the correct position in the subject's heart. If this impedance value becomes too low, this can be an indication of that the lead 142 has become disconnected from the endocardium/myocardium. A status parameter that can be useful according to the present invention is therefore the lead impedance of any leads 142 (there may be more than one lead 142 connected to the sensor interface 140).

Another important status parameter of the IMD 100 is a parameter representative of a current power level of a battery 160 in the IMD 100. This battery 160 provides the supply power to all the units of the IMD 100. If the power level becomes to low, the IMD 100 may not operate correctly and could, for instance, not have access to enough supply power for performing a desired shock therapy. The IMD 100 therefore preferably comprises a battery monitor 170 connected to the battery 160. This monitor 170 continuously, periodically or at selected time instances investigates the battery status. The monitor 170 processes this measured level for obtaining a status parameter indicative of the current power level of the battery 160.

A further example of status parameter of the present invention is a parameter indicative of the current operation mode of the IMD. An IMD can often be run in different operation modes depending on what type of therapy that is expected to be needed for the subject. Such operation mode information is a useful status parameter according to the invention. This parameter is preferably generated by the diagnostic unit 145 or some other unit in the IMD 100.

Thus, the generated parameters from the diagnostic unit 145 and the battery monitor 170 are output to the RFID programmer 120 and used by the programmer 120 when generating the programming command. During normal correct operation and without any detected potentially harmful discrepancy of diagnostic status, the RFID programmer 120 could simply generate a command that specifies that the operation status and diagnostic status are ok. In a more elaborate embodiment, the programming command of the RFID programmer 120 contains more detailed information. For example, programmer 120 could include actual information of the battery power level, lead impedance level or the actual (concentration) value determined by the diagnostic unit 145.

In a preferred embodiment, the IMD 100 includes an event detector 110 arranged for detecting an alarm or emergency event. As is illustrated in FIG. 2, the event detector 110 is connected to the diagnostic unit 145 and the battery monitor 170. The detector 110 therefore receives the output from these two units 145, 170 and uses the diagnostic parameter and/or status parameter for determining whether an alarm event is present.

The detector 110 itself or the diagnostic unit 145 and monitor 170 preferably compare the processed diagnostic or status parameters with predefined threshold values or predefined parameter patterns. In the former case, the determined diagnostic or status parameter is simply compared to the corresponding threshold value. If the parameter exceeds or falls below (depending on what quantity the parameter represents) the threshold, the detector 110 determines that there is an alarm event. It is of course possible to use multiple, i.e. at least two, threshold values for a given parameter. In such a case a first threshold could specify the borderline between normal status and “warning” status, while a second threshold specifies the borderline between warning status and alarm status. The warning status could then be an intermediate level, in which the measured parameter value starts to differ from the “normal” level but the (operation or medical) condition is not yet that severe that extensive actions must be taken. If the parameter value is changed further and enters the alarm level, emergency actions may be needed to combat the medical affection of the subject or the incorrect operation of the IMD 100.

For other types of diagnostic or status parameters, it might not be adequate to compare to simple threshold values. In such a case a morphological evaluation may be needed, for instance, by identifying certain patterns or trends in the measured parameters.

In either case, once the event detector 110 has detected an imminent alarm event, the detector 110 outputs a signal to the RFID programmer 120, causing the programmer to generate an RFID programming command representative of the detected alarm event. The programmer 120 could therefore generate a programming command comprising event information descriptive of the detected alarm event.

The IMD 100 may also have an alarm unit 180 that is used for informing the subject that an alarm event has been detected. The alarm unit 180 can run an audio and/or tactile alarm signal once the event detector 110 has detected the alarm event or when the RFID interface 130 has communicated the generated RFID programming command to the connected RFID tag. In the case of an audio signal, the subject will hear the signal and knows that something is not quite right. The subject will then, as is described in more detail further on, activate his/her portable RFID reader that interrogates newly reprogrammed RFID tag and receives information of the alarm event, which can be displayed for the subject. A tactile alarm signal could be a vibration of at least a portion of the IMD 100 outer wall that is felt by the subject.

As is illustrated in FIG. 2, the IMD 100 also preferably comprises a traditional transmitter/receiver 190 and connected antenna 195 for communicating with an external unit, such as a physician's programmer or workstation. The transmitter/receiver unit 190 comprises those functionalities required for transmitting data, in particular collected diagnostic data, and receiving data, in particular operation setting updates, such as encoder/decoder, modulator/demodulator, etc. The transmitter/receiver 190 and the connected antenna 195 can operate for communicating data through radio frequency based techniques. In an alternative embodiment, an inductive transmission technique is used. The antenna 195 could then be exchanged by an inductive coil.

It is anticipated by the present invention that the RFID programmer 120 could become operable only in response to the event detector 110 detecting an alarm event. Thus, no RFID programming commands are then generated and forwarded by the interface 130 to the RFID tag during normal operation and status. The RFID tag will therefore only be reprogrammed in an emergency situation.

However, it is also possible that the RFID programmer 120 becomes operable at other selected instances. For examples, if the operation status of the IMD 100 is changes as determined by the event detector 110 based on different input operation status parameters, the programmer 120 can generate a programming command descriptive of the IMD operation or descriptive of the change in the operation as compared to the previous IMD operation.

In another embodiment, in particular under normal operation and status, the RFID programmer 120 can become operable and reprogram the RFID tag memory periodically, such as once or twice per day or more often or seldom. The programming could then simply state “status OK”. However, also under normal operation, more detailed information could be of interest to the subject. For example, information of battery status, IMD operation mode, heart rate, etc. The programmer 120 then collects the input from other units of the IMD and composes the programming command that is sent to the RFID interface 130 for forwarding to the RFID tag.

The units 110 to 190 of the IMD 100 can be provided as hardware, software or a combination of hardware and software.

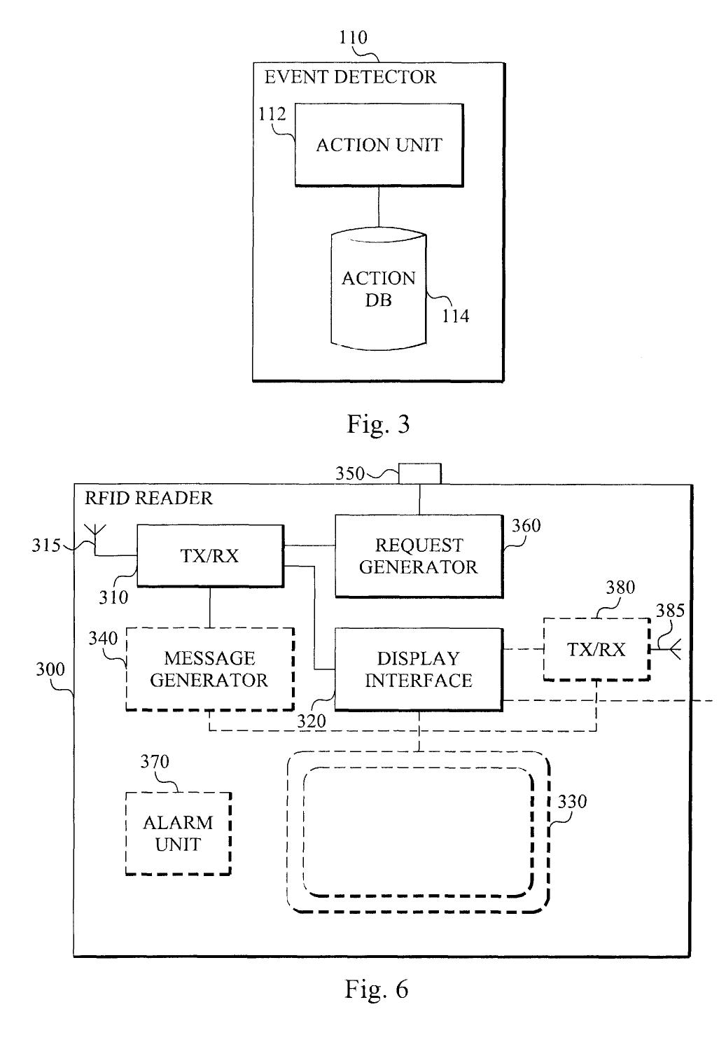

FIG. 3 is a schematic block diagram of an embodiment of the event detector 110 of FIG. 2 in more detail. This event detector 110 includes an action unit 112 arranged for generating action information based on the input diagnostic and/or operation status parameter(s). The action information is descriptive of a preferred action to be taken due to the detected alarm event. The action could, for instance, be an action intended for combating the cause of the alarm event (give first aid and/or administer some medicament) or make an emergency call for ambulance personnel, etc. The RFID programmer generates the programming command based on the derived action information.

In a preferred embodiment, the event detector 110 has access to a memory or database 114 in which multiple different predefined actions are stored. The action unit 112 uses the input data from the diagnostic unit and/or battery monitor to select a most appropriate action that should be taken in response to the current alarm event as determined based on the input data. For example, in the case a diagnostic parameter having multiple associated predefined threshold values and the measured parameter value exceeds the warning level threshold, the action could be “call your physician”. However, if the parameter level would reach the alarm level, the action could be “call 911” (or some other emergency number).

The appropriate action to be taken is therefore selected from the database 114 based on what type(s) of input parameter(s) the detector 110 has received and/or the actual value(s) of the input parameter(s). The action unit 112 thereafter generates the action information based on the selected action and forwards the action information to the RFID programmer for generation of the programming command.

The unit 112 of the event detector 110 can be provided as hardware, software or a combination of hardware and software. The units 112 and 114 may alternatively be implemented elsewhere in the IMD.

FIG. 4 is a schematic block diagram of an implantable RFID tag 200 according to the present invention. The tag 200 comprises a readable and writable memory 210, which can be reprogrammed based on programming commands received from an IMD. In FIG. 4, the tag 200 is illustrated with a wired connection to the IMD. This is a preferred implementation for a stand-alone implantable RFID tag 200. The wired connection means that the tag 200 can utilize the power supply of the IMD and therefore does not have to contain a separate battery or other power source. Furthermore, no equipment for conducting wireless communication with the IMD is needed. In total this enables smaller and cheaper RFID tag designs. The tag 200 could be implanted onto the housing of the IMD or, as is illustrated in FIG. 1, within a short distance from the IMD. However, it is actually possible to provide the IMD and the tag 200 at more mutually remote locations inside the subject body as long as it is possible to have a wired connection therebetween.

In an alternative approach, the RFID tag 200 communicates wirelessly with the IMD. The tag 200 therefore contains an own battery and receiver with connected antenna for receiving the programming commands. The antenna and receiver preferably operates according to short range radio frequency transmission techniques. However, also other wireless techniques that are able to communicate data at a short distance, typically up to a few centimeters or one or few decimeters, inside a subject body can be used according to the present invention.

The RFID tag 200 also includes a transmitter/receiver unit 225 with connected antenna 220 for communicating with an external RFID reader. The antenna 225 and receiver chain of the unit 220 are adapted for receiving an information request or memory interrogation from the RFID reader. The content of the tag memory 210 is then compiled to a message sent by the antenna and the transmitter chain of the unit 220 to the reader.

In a preferred embodiment of the invention, the tag memory 210 comprises a readable and writable, i.e. programmable, memory portion 212 and a readable memory portion 214 with a predefined memory content 214. The programming commands from the IMD then brings about a reprogramming of the content of the programmable memory portion 214 but not the predefined memory portion 214. However, at a memory interrogation, the contents of both these memory portions 212, 214 are preferably returned to the RFID reader.

The predefined memory 214 comprises such information that does not change during the operation of the IMD or over, at least a limited, period of time. This information can relate to the particular subject, such as his/her name and/or identification information, such as social security number. Information relating to the subject's physician, such as name, contact information (address, phone number, fax number, e-mail address, etc.), can also be useful predefined information that is entered in the predefined memory portion 214. A further example is information relating to the implantable medical device. This type of information includes the device manufacturer, model number and serial number.

It could actually be possible, in a particular implementation, to reprogram even the predefined memory portion 214. However, in such a case, the IMD is only able to dynamically program the programmable memory portion 212, whereas the predefined memory portion 214 is only programmable through telemetry with an external communication unit, such as programmer or PDA. To prevent fraudulent reprogramming of the predefined memory 214, an authentication procedure and/or encoded data transmission can be used to only allow an authorized external unit to reprogram the memory portion 214. This external unit is preferably housed by the subject him/herself or his/her physician. The reprogramming could be useful in the case of change of physician, etc.

In contrast, the content of the programmable memory portion 212 becomes updated typically every time the RFID tag 200 receives a programming command from the IMD. The information contained in this memory portion 212 therefore includes information of the measured diagnostic and/or operation status parameter(s); reflects a detected alarm, such as by specifying a detected medical condition or deleterious operation status; and/or includes instructions of a desired action to be taken.

An example of possible information contained in the tag memory 210 is given below:

Name: John Smith

Physician: Dr. Dowe at Cleveland Clinic

IMD model: Frontier II CRT-P

Heart rate: 67

Condition: Atrial Fibrillation

Therapy: 1 shock at 24 J

Mode: DDD

Time: 12:05 Jan. 4, 2007

Action: Call 911, read the information above to the emergency operator.

The first three lines specify such information that can be stored in the predefined memory portion 214. The fourth line is a diagnostic parameter that is collected by the diagnostic unit. The fifth line contains information of a diagnosed medical condition of the subject as determined based on a comparison of measured diagnostic parameters with different threshold values. The sixth and seventh line contain information of the operation status of the IMD by, in this case, specifying the type of therapy applied due to the detected medical condition and in what operation mode the IMD is currently running. The eights line comprises time information on when the information was entered the RFID tag memory. The last line above includes action information that has been selected by the event detector of the IMD based on the collected diagnostic and operation status parameters.

As there is a vast amount of different medical conditions and operation malfunctions possible and therefore also a vast amount actions and status parameters that could be of relevance according to the invention, it is not feasible to list all such possible examples of information that be stored in or entered into the memory 210 of the RFID tag. However, the above presented examples and discussions of memory contents can be seen as illustrative teachings for exemplifying how the present invention can be utilized in practice.

In order to provide an enhanced security aspect, in particular for an RFID tag 200 communicating wirelessly with the IMD, the tag memory 210, or at least the programmable portion 212 thereof, could be configured to only be programmable through the IMD. This can be solved by an encoded transmission, in which the RFID tag 200 and IMD are provided with mutual encryption/decryption key(s) or through a more elaborated authentication procedure. However, in the case of a wired connection between the RFID tag 200 and the IMD, this security aspect can be fulfilled by only allowing the tag memory 210 to be reprogrammable using commands originating from the tag-IMD wired connection.

The unit 220 of the RFID tag 200 can be provided as hardware, software or a combination of hardware and software.

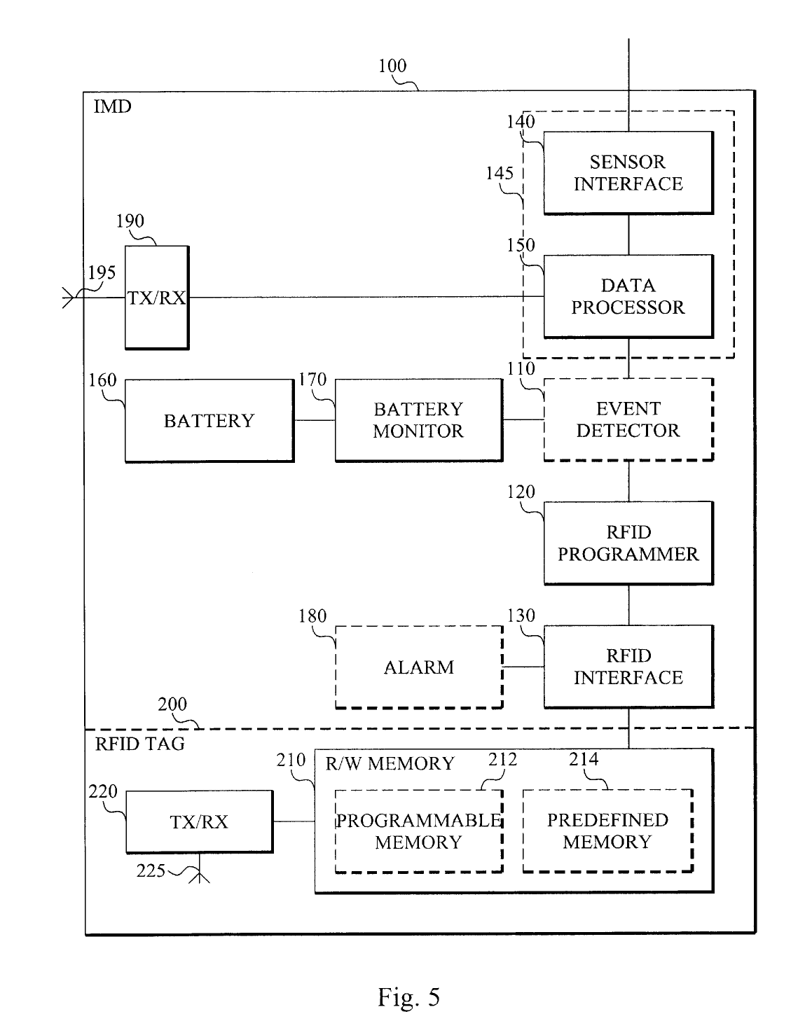

In the previous embodiments, the IMD and RFID tag has been separate units hardwired or wirelessly connected to each other. However, it is actually possible to provide an IMD with an RFID tag arranged onto or even inside the IMD housing. Such an IMD embodiment 100 is illustrated in FIG. 5. This IMD embodiment 100 basically includes a combination of those units illustrated in FIG. 2 and described above in connection with that figure and those units illustrated in FIG. 4 and presented in connection with the RFID tag discussion. The operation of the respective units is as previously described and is not repeated herein. In this embodiment, the units 210, 220, 225 of the RFID tag 200 can utilize the power supply of the IMD battery 160. This embodiment may also have the advantage that it larger memory sizes could be used by the RFID tag 200 due to a possible larger space inside the IMD 100 as compared to a separate tag 200. The tag 200 can also be protected from the body environment by the housing of the IMD 100 or a separate housing attached to the IMD housing.

The units 110 to 190 and 220 of the combined IMD and RFID tag device can be provided as hardware, software or a combination of hardware and software.

FIG. 6 is a schematic block diagram of an embodiment of a RFID reader or interrogator 300 or a portable smart card comprising such an RFID reader functionality according to the present invention. The RFID reader 300 comprises a transmitter/receiver unit 310 with a connected antenna 315. The transmitter chain of the unit 310 transmits, using the antenna 315, an information request to the implantable RFID tag. The tag responds by forwarding the information in the tag memory to the antenna 315 and receiver chain of the unit 310. The received information is forwarded to a display interface 320. This interface 320 is, in operation, connected (wired or wireless) with a display screen 330 and displays the received information thereon. In a first embodiment, the reader 300 includes a display screen 330, such as a liquid crystal display (LCD) screen. In a second embodiment, the display interface 320 is connected to an external display screen through a wired connection as is schematically illustrated in FIG. 6. This external screen could be a display screen of so-called smart clothing. The screen is therefore connected to the fabric of, for example, a jacket worn by the subject. A further embodiment is to have a further transmitter 380 with a connected antenna 385. The display interface 320 then forwards the received information from the RFID tag to the transmitter for wireless transmission, using e.g. RF, Bluetooth® or IR based transmission techniques, to another external device having a display capability. This external device could be a portable user terminal, such as mobile telephone or personal digital assistance (PDA), carried by the subject. In these two latter embodiments, the size of the reader can be kept small as no internal display screen is needed.

In a preferred implementation, the RFID reader 300 comprises a user input 350, schematically exemplified by a push button in FIG. 6. This user input 350 is to be activated once the RFID reader 300 should interrogate the implantable RFID tag. The user input 350 could therefore, for instance, be a push button on the reader 300 or a dedicated area of the touchable (LCD) screen 330. Once the user input 350 has been activated, a connected request generator 360 generates the information request that is transmitted by the transmitter 310 and antenna 315 to the RFID tag.

Alternatively, or preferably in addition, the request generator 360 can be configured for periodically, such as once or twice per day or more often/seldom, automatically generate a tag memory interrogation that is transmitted by the transmitter 310. The subject can then automatically get information of the IMD operation status and/or his/her diagnostic status.

The RFID reader 300 may optionally contain a message generator 340. This generator 340 is connected to the receiver 310 arranged for receiving memory data from the implantable RFID tag. The content of the received transmission is forwarded to the message generator 340 that compiles an alarm message based on the information. This alarm message could basically contain the same information that is presented on the display screen or at least a portion thereof. The generated alarm message is forwarded to the transmitter 380 that performs communication with external units. In this case, the transmitter 380 forwards the alarm message via a communication network, such as wireless local area network (WLAN) or radio-based cellular communications network, to a predefined healthcare facility. This facility could be an emergency central managing emergency calls. Another suitable example could be the healthcare facility at which the subject's physician is working. In an alternative approach, the transmitter 380 sends the alarm message to a portable communications terminal carried by the subject, which in turn forwards the message via the network to the emergency center.

The RFID reader 300 may optionally contain an alarm unit 370 that is responsive to the reception of the requested information from the RFID tag by the receiver 310. This alarm unit 370 could be arranged for running a visual, audio and/or tactile alarm notifying the subject that information has been received and is now available on the display screen 330. The alarm may also be useful in the case the subject is unconscious or otherwise so severely affected that he/she cannot himself/herself view the screen 330. The alarm will instead attract the attention of people in the surroundings that will therefore see the information presented on the screen 330 and can take the appropriate action based on the displayed information. A visual alarm 370 could be the blinking or constant activation of a dedicated lamp on the reader 300 or actually of the display screen 330. An audio alarm 370 can be realized by a loudspeaker that can run a pre-defined alarm signal. A vibration unit is an example of a tactile alarm device 370 of the invention.

It is anticipated by the present invention that the predefined memory portion disclosed in FIGS. 5 and 6 and implemented in the RFID tag could alternatively be provided in the RFID reader 300. Thus, the content of that memory portion does not need to be updated by the IMD so it can therefore instead be included in the reader 300 to save space of the implantable RFID tag.

It could be possible that the subject becomes unconscious or so severely affected by the cause of the detected alarm event that he/she cannot activate the user input 350. In such a case, the reader backside could include information urging a person finding the reader to active the user input 350. The text could, for example, be “Please press the button and take the actions displayed on the screen”. If the request generator 360 is configured for automatically generating tag memory interrogations, descriptive information can automatically be received by the RFID reader 300 and displayed on the screen 330. In such a case, there is a trade-off between interrogating the memory to often, which drains the power supply of the reader 300, and interrogation to seldom, in which case no updated useful information is available on the screen 330. However, the automatic tag interrogation can advantageously be complemented with a user activation via the push button 350.

The units 310, 320 and 340 to 380 to 190 and 220 of the RFID reader 300 can be provided as hardware, software or a combination of hardware and software.

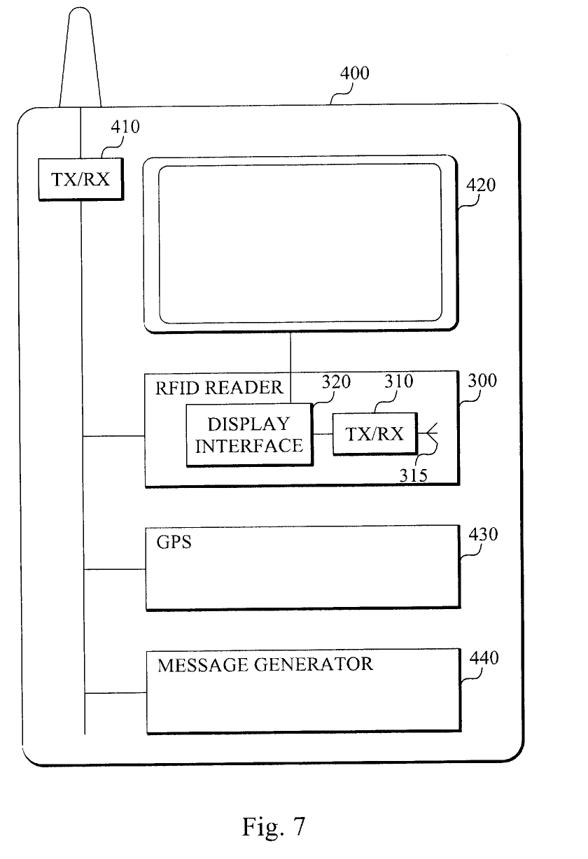

FIG. 7 is a schematic block diagram of an embodiment of a portable communications terminal 400 that can form a part of the condition system of the present invention. In FIG. 7 the communications terminal 400 has been exemplified by a mobile telephone but could instead be a PDA or other terminal capable of performing remote communication over a communication network.

In FIG. 7, the terminal comprises a RFID reader 300 according to the present invention. This reader 300 comprises the previously described transmitter/receiver unit 310 with connected antenna 315 for interrogating the implantable RFID tag and the display interface 320. The reader 300 could also include the request generator and/or alarm unit disclosed in FIG. 6. The user input connected to the request generator could be one of the keys of the terminal (not illustrated) or the touch sensitive (LCD) screen 420.

The terminal 400 preferably also comprises a positioning unit 430 for generating data descriptive of a current position of the terminal. This unit 430 could use the Global Positioning System (GPS) or some other Global Navigation Satellite System (GNSS) that operates as a GPS/GNSS receiver and processor for providing location information based on the received GPS data. In an alternative embodiment, the unit 430 could use information from the communication network in the location determining process, such as through the well-known triangulation technique.

A message generator 440 is arranged in the terminal 400 connected to the RFID reader 300 and the preferred positioning unit 430. The generator 440 receives the information requested by the RFID reader 300 from the tag. In addition, the generator 440 receives or requests position information from the positioning unit 430. This input data is employed by the generator 440 for compiling a message that is automatically sent to a predefined healthcare facility. This means that the facility will receive information descriptive of the alarm event, such as the description of the detected severe medical condition in the IMD subject, together with information of the subject and IMD. This information is useful for the receiving emergency personnel as they know what has happened to the subject once they reach him/her and also that he/she has an implanted medical device. The provided position information facilitates a quick location of the subject, reducing the time until medical personnel will reach the subject. The predefined healthcare facility could be an emergency center, a defined hospital or the mobile telephone, PDA or computer of the subject's physician.

The received information from the RFID tag is also preferably displayed in the screen 420 of the terminal 400 allowing the subject or other persons finding the subject to see information.

It is anticipated by the present invention that the predefined memory portion disclosed in FIGS. 5 and 6 and implemented in the RFID tag could alternatively be provided in the terminal 400. Thus, the content of that memory portion does not need to be updated by the IMD so it can therefore instead be included in the terminal 400 to save space of the implantable RFID tag.

The units 410, 430 and 440 of the communications terminal 400 can be provided as hardware, software or a combination of hardware and software.

In an alternative embodiment of the condition system of the present invention is, as illustrated in FIG. 1, the RFID reader 300 not implemented in the communications terminal 400. Instead the reader 300 includes a transmitter and antenna for wirelessly communicate with the terminal 400, such as using RF, Bluetooth® or IR based techniques. This means that the terminal 400 comprises a receiver adapted for receiving the alarm message generated by the message generator of the RFID reader 300. In still another embodiment, the reader 300 and terminal 400 are separate entities but connected by a wire.

The RFID tag and reader of the present invention preferably operates in the Ultra High Frequency (UHF) range of 300 MHz to 3 GHz and more preferably in the UHF sub-band of 850 to 950 MHz that is used by the EPC (Electronic Product Code) standard UHF RFID tags.

Implantable RFID tags are known in the art, for example as disclosed in U.S. Patent Application Publication Nos. 2005/0247319 and 2006/0212096. There are several implantable RFID tags available today both for humans and animals, including implantable RFID tags from VeriChip.

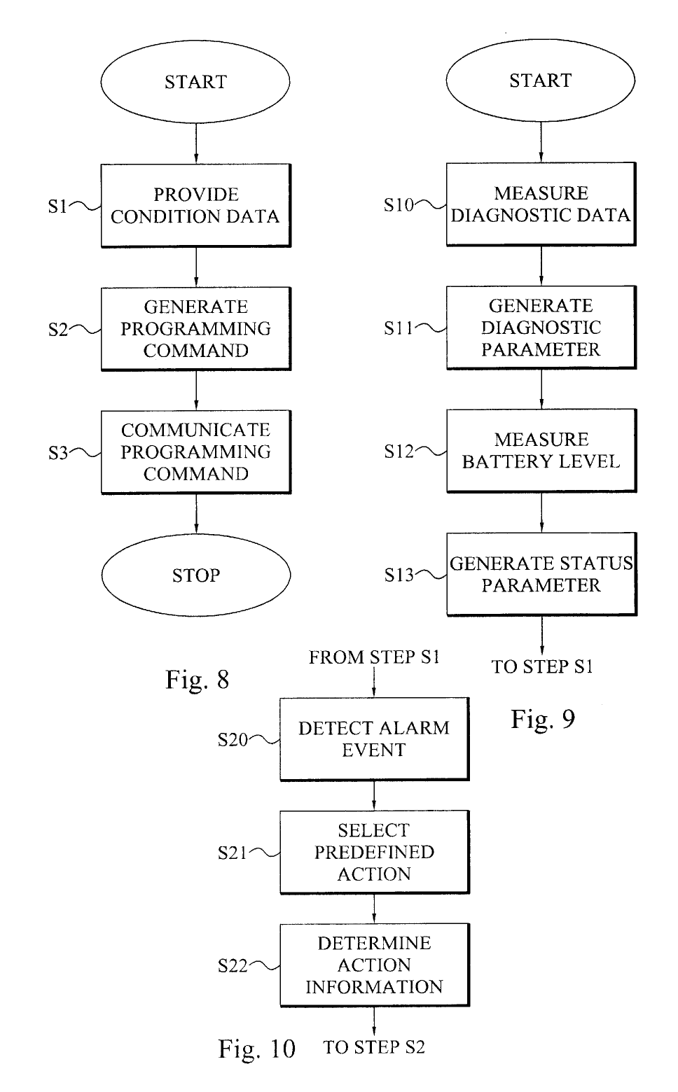

FIG. 8 is a flow diagram of a condition method operable in the condition system of the present invention comprising an implantable medical device and an implantable RFID tag. The method starts in step S1, where the IMD provides condition data, such as data descriptive of a current medical status of the subject and/or operation status of the IMD. The IMD generates a programming command based on this provided data in step S2. The command is then communicated to the RFID tag implemented inside the IMD or wirelessly or hardwired connected to the IMD in step S3. There the readable and writable memory of the tag is reprogrammed based on the received programming command. Thus, the content of the tag memory will, after the operation of step S3, contain information of the current diagnostic patient status and/or IMD operation status. The method then ends.

FIG. 9 is a flow diagram illustrating additional steps of the condition method of FIG. 8. The method starts in step S10 where the IMD performs diagnostic measurements in the subject body using at least one internal or connected sensor. The measurement data is processed in step S11 for generating at least one diagnostic parameter that is reflective of a medical status or condition of the subject.

In a next step S12 the current battery level of the IMD is measured for generating a status parameter in step S13 representative of the current power level or remaining power of the IMD battery. The method then continues to step S1 where the diagnostic parameter and the status parameter can be used for providing the condition data, based on which the programming command is generated.

It is anticipated by the present invention that in a first embodiment only the steps S10 and S11 of the FIG. 9 are conducted in the IMD. This means that the programming command will only be based on at least one diagnostic parameter. Correspondingly, in a second embodiment only the steps S12 and S13 of FIG. 9 are performed. The programming command will then be reflective of the operation status of the IMD. Instead of steps S12 and S13 or as a complement thereof, other operation status parameters can be generated in the IMD, such as the previously described lead impedance and IMD operation mode.

FIG. 10 is a flow diagram of additional steps of the condition method of FIG. 8. The method continues from step S1 of FIG. 8. In a next step S20, the IMD processes the diagnostic parameter(s) and/or status parameter(s) for the purpose of detecting any alarm or emergency event. This alarm event corresponds to the detection of an abnormal IMD operation or the detection of a non-normal (severe) medical condition in the subject. The programming command is then generated to be descriptive of the alarm event. In an embodiment this is performed by selecting a predefined action based on the diagnostic and/or status parameter in step S21. This action can be an action to be taken by the subject or another person to combat the cause of the alarm event or to call an emergency center. In such a case, there are several different predefined actions and the most appropriate to take in the current situation is determined based on the diagnostic and/or status parameter. Information descriptive of the selected action is determined in step S22 and will be used in step S2 of FIG. 8 for generating the programming command.

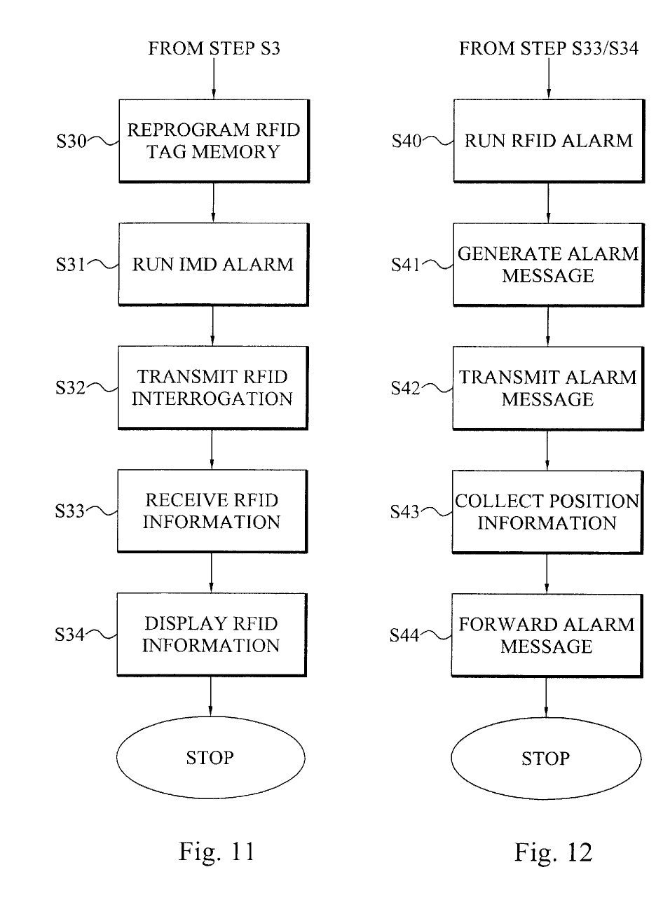

FIG. 11 is a flow diagram illustrating additional steps of condition method of FIG. 8. The method continues from step S3 of FIG. 8. In a next step S30, the memory of the implantable RFID tag is reprogrammed based on the received programming command from the IMD. In an optional step S31, the IMD runs an audio and/or tactile alarm signal once the new programming command has been generated or transmitted to the RFID tag. This alarm signal urges the subject to interrogate the RFID tag in step S32 using the portable RFID reader. Thus, the subject or some other person actives a user input of the RFID reader for generating and transmitting an information request to the implantable RFID tag. The tag responds by transmitting data corresponding to the information stored in its readable and writable memory. This data is received by the RFID reader in step S33. The data is forwarded to a display interface for display on a screen of the RFID reader or an external screen, such as on a portable communication unit, separate display unit or screen attached to an article of clothing, where the data is displayed in step S34.

Any person reading the display can then get information of what has happened to the subject or the IMD and preferably what to do. Furthermore, information of the subject, the IMD and any contact information to physicians etc. can also be presented on the screen. The information is a valuable source for the medical personal first reaching the subject as correct actions or treatment can be initiated directly without any extensive diagnosis of the subject.

Also automatic and period generation and transmission of tag information requests can be used as previously described.

FIG. 12 is a flow diagram illustrating additional steps of the condition method of FIG. 8. The method continues from step S33 or S34 of FIG. 11. In a next step S40, the RFID reader runs a visual, audio and/or tactile alarm informing the subject that information from the RFID tag has now been received and is visible on the display screen. The alarm can also be used for attracting persons in the vicinity of the patient to look on the display screen and take actions accordingly if the subject is not able to do this himself/herself.

In a next step S41, the RFID reader compiles an alarm message based on the received information from the tag memory in step S41. The message is forwarded or transmitted to a portable communications terminal carried by the subject in step S42. If possible, the terminal collects position information using, for instance, the GPS system in step S43. In a next step S44, a message containing information from the received alarm message and preferably the position information is compiled and transmitted to a predefined healthcare facility, such as an emergency center. The healthcare facility can then send ambulance personnel to the subject using the location information. In addition, the personnel will already have access to data of the subject, the IMD and current medical condition of the subject as included in the message sent in step S44. The method then ends.

With reference to FIG. 1, in a typical use scenario the subject 10 feels that something is not quite right or the IMD 100 runs an alarm. The IMD 100 has previously collected data descriptive of the abnormal condition and determined that something is wrong with the subject 10 or the IMD 100. The IMD 100 has also activated the connected RFID tag 200 and written information descriptive of the detected condition into the tag memory.

The subject 10 has been instructed in the case of an alarm to fetch the portable RFID reader 300 and active the user input thereon. The reader 300 makes an interrogation of the tag memory and reads the information from the RFID tag 200. The collected information is displayed on the screen of the reader 300. The subject 10 reads the information from the display and acts accordingly.

For example, the subject 10 can give first aid to himself/herself, take certain medicaments as notified on the screen, contact an emergency center or his/her physician, depending on the severity of the alarm.

In addition, the reader 300 could include far field communication equipment to automatically send a message to the emergency central, where the message includes the information received from the RFID tag.

Alternatively, the reader 300 automatically sends to a message to a mobile communications terminal 400 carried by the subject 10. The terminal 400 has GPS capabilities or other mobile positioning capabilities. The terminal 400 sends an automatic message to the emergency central, where the message includes the information received from the RFID tag and the position information.

In addition, or alternatively, the RFID reader 300 actives an alarm attracting people in the vicinity and helping them in finding the reader 300. These persons will read the instructions on the display and can take the appropriate actions based on the displayed instructions.

In addition, or alternatively, the subject 10 is wearing a smart jacket with display capabilities. The RFID reader 300 then sends instructions to the display screen of the jacket, where the information from the RFID tag is displayed. The reader 300 could also run its alarm for attracting people to read the instructions on the jacket.

It is anticipated by the present invention that in a general aspect, IMD can generate a programming command based on any information or data generated by the IMD or received from units connected thereto. The command is then transmitted to the implantable RFID tag and is used for reprogramming the tag memory.

It will be understood by a person skilled in the art that various modifications and changes may be made to the present invention without departure from the scope thereof, which is defined by the appended claims.

Although modifications and changes may be suggested by those skilled in the art, it is the intention of the inventors to embody within the patent warranted heron all changes and modifications as reasonably and properly come within the scope of their contribution to the art.

We claim as our invention therefor:

1. An implantable medical device comprising:
a radio frequency identification, RFID, programmer that generates a programming command representative of a diagnostic status of a subject and/or an operation status of said implantable medical device; and
an RFID interface that communicates said programming command to a programmable RFID tag.

2. The implantable medical device according to claim 1, further comprising an event detector that detects an alarm event based on a diagnostic parameter indicative of said diagnostic status and/or a status parameter indicative of said operation status, wherein said RFID programmer is arranged for generating said programming command representative of said alarm event detected by said event detector.

3. The implantable medical device according to claim 2, wherein said RFID programmer generates a programming command comprising event information descriptive of said alarm event detected by said event detector.

4. The implantable medical device according to claim 3, wherein said event information comprises at least one of:
status information of said implantable medical device as determined by said event detector based on said status parameter;
condition information descriptive of a medical condition of said subject as determined by said event detector based on said diagnostic parameter; and
action information descriptive of an action to be taken in response to said alarm event as determined by said event detector based on said status parameter and/or said diagnostic parameter.

5. The implantable medical device according to claim 2, wherein said event detector comprises an action unit arranged for generating, based on said diagnostic parameter and/or said status parameter, action information descriptive of an action to be taken due to said alarm event and said RFID programmer generating said programming command based on said action information.

6. The implantable medical device according to claim 5, wherein said action unit is configured to i) select, based on said diagnostic parameter and/or said status parameter, said action from a set of predefined different actions and ii) generate said action information based on said selected action.

7. The implantable medical device according to claim 2, further comprising a diagnostic unit comprising:
a sensor interface for connection, in operation, to a sensor and for receiving measurement data collected by said sensor;
a data processor that generates said diagnostic parameter by processing said measurement data received by said sensor interface.

8. The implantable medical device according to claim 2, further comprising:
a battery supplying power to said implantable medical device (100); and
a battery monitor that generates said status parameter based on a current power level of said battery.

9. The implantable medical device according to claim 1, further comprising an alarm unit that generates an audio and/or tactile alarm signal in response to said RFID interface communicating said programming command to said programmable RFID tag.

10. A condition system comprising:
an implantable programmable radio frequency identification, RFID, tag comprising a read and writable memory; and
an implantable medical device comprising:
an RFID programmer that generates a programming command representative of a diagnostic status of a subject and/or an operation status of said implantable medical device; and
an RFID interface that communicates said programming command to said RFID tag for reprogramming said readable and writable memory (210).

11. The system according to claim 10, wherein said readable and writable memory comprises:
a first memory portion with predefined information; and
a second memory portion being reprogrammable by said programming command.

12. The system according to claim 11, wherein said predefined information comprises at least one of:
information relating to said subject, such as name and social security number;
information relating to a physician of said subject, such as physician name and contact information; and
information relating to said implantable medical device, such as device manufacturer, model number and serial number.

13. The system according to claim 10, further comprising an RFID reader comprising:
a transmitter that transmits an information request to said RFID tag;
a receiver that receives information stored in said read and writable memory of said RFID tag; and
a display interface for connection, in operation, with a display screen and for forwarding said received information for display on said display screen.

14. The system according to claim 13, wherein said RFID reader further comprises:
a user input; and
a request generator that generates said information request in response to an activation of said user input.

15. The system according to claim 13, wherein said RFID reader further comprises:
a message generator that generates an alarm message based on said received information; and
a transmitter for transmitting said alarm message to a predefined healthcare facility.

16. The system according to claim 13, wherein said RFID reader further comprises an alarm unit that emits a visual, audio and/or tactile alarm signal based on said receiver receiving said information.

17. The system according to claim 13, further comprising a mobile communications terminal comprising:
a receiver that receives an alarm message from said RFID reader generated based on said received information;
a positioning unit that generates data descriptive of a current position of said mobile communications terminal; and
a transmitter for transmitting information contained in said alarm message and said data to a predefined healthcare facility.

18. The system according to claim 13, further comprising a smart clothing comprising said display screen.

19. The system according to claim 10, wherein said implantable medical device comprises an event detector that detects an alarm event based on a diagnostic parameter indicative of said diagnostic status or a status parameter indicative of said operation status, wherein said RFID programmer is arranged for generating said programming command representative of said alarm event detected by said event detector.

20. A condition method operable in a system comprising an implantable medical device and an implantable programmable radio frequency identification, RFID, tag, said method comprising, in said implantable medical device:
generating a programming command representative of a diagnostic status of a subject and/or an operation status of said implantable medical device; and
communicating said programming command to said programmable RFID tag.

21. The method according to claim 20, further comprising detecting an alarm event based on a diagnostic parameter indicative of said diagnostic status or a status parameter indicative of said operation status.

22. The method according to claim 21, wherein said generating step comprises:
determining, based on said diagnostic parameter and/or said status parameter, action information descriptive of an action to be taken due to said alarm event; and
generating said programming command based on said action information.

23. The method according to claim 22, wherein said determining step comprises:
selecting, based on said diagnostic parameter and/or said status parameter, said action from a set of predefined different actions; and
determining said action information based on said selected action.

24. The method according to claim 21, further comprising:
measuring diagnostic data in said subject; and
said implantable medical device generating said diagnostic parameter by processing said measured diagnostic data.

25. The method according to claim 21, further comprising generating said status parameter based on a current power level of a battery supplying power to said implantable medical device.

26. The method according to claim 20, further comprising said implantable medical device generating an audio and/or tactile alarm signal in response to communication said programming command to said RFID tag.

27. The method according to claim 20, further comprising reprogramming a read and writable memory of said RFID tag based on said programming command.

28. The method according to claim 20, further comprising the steps of, in an RFID reader:
transmitting an information request to said RFID tag;
receiving information stored in said read and writable memory of said RFID tag; and
forwarding said received information to for display on a display screen.

29. The method according to claim 28, comprising performing said transmitting step in response to activation of a user input of said RFID reader.

30. The method according to claim 28, further comprising:
generating an alarm message based on said received information; and
transmitting said alarm message to a predefined healthcare facility.

31. The method according to claim 28, further comprising said RFID reader generating a visual, audio and/or tactile alarm signal based on said reception of said information stored in said read and writable memory.

32. The method according to claim 28, further comprising, in a mobile communications terminal:
receiving an alarm message from said RFID reader generated based on said information received by said RFID reader from said read and writable memory;
generating data descriptive of a current position of said mobile communications terminal; and
transmitting information contained in said alarm message and said data to a predefined healthcare facility.
